(19) United States
(12) Patent Application Publication
(10) Pub. No.: US 2014/0052111 A1
(43) Pub. Date: Feb. 20, 2014

(54) FLOCKED MEDICAL TUBE

(71) Applicant: Aesculap AG, Tuttlingen (DE)

(72) Inventors: Erich Odermatt, Schaffhausen (CH); Wolfgang Abele, Tuttlingen (DE)

(73) Assignee: Aesculap AG, Tuttlingen (DE)

(21) Appl. No.: 13/968,758

(22) Filed: Aug. 16, 2013

(30) Foreign Application Priority Data
Aug. 17, 2012 (DE) 10 2012 214 640.7

(51) Int. Cl. A61M 27/00

(52) U.S. Cl. CPC A61M 27/00
USPC 604/543; 427/2.28

(57) ABSTRACT

A medical tube, especially for use in vacuum therapy, preferably endoluminal vacuum therapy, comprising at least one tube body and a flock material is described. A process for producing a medical tube comprises: coating the surface of at least one tube body with an adhesive at least partially, especially partially or completely, and flocking the adhesive-coated tube body with a flock material, while the adhesive is moist, adhesive or sticky, or after activating the adhesive. Alternatively, a process for producing a medical tube comprises: at least partial transformation of at least one tube body into an adhesive or sticky state and flocking of the at least partially adhesive or sticky tube body with a flock material. In addition, a drainage system, especially in the form of a kit is described.

### TECHNICAL FIELD

This disclosure relates to a medical tube with a flock material, a process for producing said tube and a drainage system.

### BACKGROUND

Medical drainage tubes for vacuum therapy of wounds are known. Tubes of this kind are described for example in WO 2006/005939 A1 and WO 2009/140376 A1.

Drainage tubes are used in endoluminal vacuum therapy, usually together with a sponge, which is provided for absorbing body fluids, for example wound fluids.

This is, however, associated with some disadvantages.

Thus, the sponge together with the tube must as a rule be changed every three days, as otherwise there is a risk of tissue adhesions, which make it difficult to remove the sponge, cause injuries and moreover can be painful for the patient.

In addition, as wound healing progresses, the sponge size must as a rule be adjusted, which on removal can lead to a (re-) opening-up of a wound.

Another disadvantage is that passage of the sponge can prove difficult, especially through small body orifices. An additional complication is that the sponges usually employed often only possess moderate to poor sliding properties along tissue surfaces.

Another problem relates to the handling of the sponge. Depending on the material of the sponge, its structural integrity can be destroyed if for example it is grasped and moved with a guiding instrument, for example forceps.

In addition, the generally open-pore configuration of the sponge can be a disadvantage, as there is always a basic risk of clogging of the pores and a consequent decrease in absorption capacity of the sponge. It may therefore even be necessary to use several sponges.

Another possible problem is the joint between sponge and tube. Usually the sponge is glued to the tube. This can result in undesirable thickening in the region of the glued points, which can impair fluid communication between sponge and tube.

Finally, the sponge together with the tube must usually be trimmed specifically for the patient.

### SUMMARY

We provide a medical tube including at least one tube body and a flock material.

We also provide a process for producing a medical tube including coating the surface of at least one tube body with an adhesive at least partially, especially partially or completely, and flocking the adhesive-coated tube body with a flock material, while the adhesive is moist, adhesive or sticky, or after activating the adhesive.

We further provide a process for producing a medical tube including at least partial transformation of at least one tube body into an adhesive or sticky state and flocking of the at least partially adhesive or sticky tube body with a flock material.

We also provide a drainage system including at least one medical tube and at least one further component that is selected from the group consisting of sponge, inserting instrument, source of vacuum or negative pressure, redrop bottle, guide wire, spiral mandrin and combinations thereof.

### BRIEF DESCRIPTION OF THE DRAWINGS

FIG. 1a is a schematic diagram depicting a first example of a medical tube.

FIG. 1b is a schematic diagram depicting a second example of a medical tube.

FIG. 1c is a schematic diagram depicting a third example of a medical tube.

FIG. 1d is a schematic diagram depicting a fourth example of a medical tube.

### DETAILED DESCRIPTION

This disclosure provides a technical solution that addresses the problems mentioned above.

In a first aspect, this disclosure relates to a medical tube. The tube is intended in particular for use in vacuum therapy, preferably endoluminal vacuum therapy.

The tube according to this disclosure comprises at least one tube body and is characterized in particular in that moreover it has a flock material.

In other words this disclosure provides a flocked tube for use in medicine.

The medical tube can basically be selected from the group comprising drainage tube, rinsing tube, disinfecting tube (tube for supplying a disinfectant), tube for monitoring or control of negative pressure or vacuum, monitoring a temperature and/or for administering therapeutic agents.

In especially preferred examples, the medical tube of this disclosure is a medical drainage tube and correspondingly the at least one tube body is at least one drainage tube body.

In other words the tube according to this disclosure is preferably a tube for leading away or aspirating especially pathological and/or increased body fluids (drainage).

The term “at least one tube body” or “at least one drainage tube body”, in the sense of this disclosure, comprises a tube body or drainage tube body or a plurality of tube bodies or drainage tube bodies, i.e. two or more tube bodies or drainage tube bodies.

The tube body of the tube according to this disclosure preferably defines a dimensionally stable lumen or a dimensionally stable cavity, which is bounded by a wall of the tube body. This has the advantage that on applying a negative pressure or vacuum, collapse of the tube body can be avoided.

The ends of the tube body are preferably configured open.

However, it can also be envisaged that one of the ends, preferably an end of the tube body that is flocked, i.e. has the flock material, is sealed, for example glued or welded. This can be of advantage mainly in the case of a drainage tube, for increasing its suction power.

Furthermore, it can be envisaged that the tube body does not define a lumen or a cavity until a medium flows through it, in particular a fluid, for example rinsing fluid, disinfecting fluid, wound fluid etc. or it is supplied with a gas, for example compressed air. In other words the tube body can originally be of flat configuration.

The term “flock material” is to be understood, in the sense of this disclosure, as a material that can be applied on the surface of the tube body by means of a flocking technique, especially using an adhesive.

The tube makes the use of a sponge, especially of the type described at the beginning, superfluous, so that the disadvantages described in the introduction in connection with sponges can be avoided.

Thus, in particular, the tube allows a prolonged residence time in a patient's body, because (in contrast to the case of a sponge) there are no undercuts or pores, into which the body tissue could grow, with formation of tissue adhesions.

The risk of tissue adhesions can moreover be minimized because as a rule the flock material offers less area for the growth or ingrowth of cells or tissues and in particular exerts a separating function against cells or tissues. In addition, the flock material can be selected on the basis that, owing to its flexibility, it can be pulled off of a tissue more easily.

Another advantage is that the tube can have much smaller dimensions than the sponges known from the prior art. Therefore the tube can also be moved through small body orifices without major problems and in particular without aids.

Furthermore, placement of the tube is possible by means of much smaller trocars and/or incisions. As a result, placement and removal of the tube is simplified considerably and moreover is less traumatic for the patient.

For filling larger body lumina or cavities, there is in addition the possibility of transforming the tube into a ball following its placement in a body lumen or cavity.

A further advantage is in particular that the flock material increases the surface area of the tube, so that much less tube length has to be pushed into a body lumen or a body cavity.

Another advantage is that as a rule the flock material is mechanically more stable than a sponge, so that the risk of mechanical damage, for example on gripping with forceps, is greatly reduced.

As the flocked tube, in contrast to the sponges known from the prior art, does not have any cavities in the sense of pores, the risk of clogging is also greatly reduced.

Another advantage is that the flock material can endow the tube with better sliding properties overall, so that the use of lubricating gels, especially when using small trocars or overtubes, is no longer necessary and direct placement can take place using forceps or in the endoscope working channel.

Depending on the flock material and/or its depth of penetration into the tube body or into an adhesive layer formed on the surface of the tube body, the properties, especially mechanical properties and/or sliding properties, of the tube can be adapted in a targeted manner, i.e. specifically for the indication and/or for the patient.

A further advantage is that, in contrast to gluing a sponge to a tube body, flocking of the tube body can be automated.

Finally, the flocked tube can be tailored or manufactured simply and comfortably with respect to a patient's specific needs.

As a rule—as will be described in more detail below—the flock material is joined directly or via an adhesive layer to the tube body or to the surface, especially inside and/or outside surface, preferably outside surface, of the tube body. Joining is as a rule based on adhesive forces.

When the surface of the tube body is referred to in the following, this is to be understood as the inside and/or outside surface, preferably the outside surface, of the tube body.

In a preferred example, the flock material is joined to the tube body or its surface via an adhesive layer at least partially covering the surface of the tube body.

The adhesive layer can cover the surface of the tube body basically completely or on the whole area, apart from any openings in the wall of the tube body.

It is, however, preferable if the adhesive layer covers the surface of the tube body only partially or on a partial area.

In particular, the adhesive layer can cover the surface of at least one, especially of only one, end of the tube body.

The adhesive layer can cover the surface of the tube body, preferably starting from at least one end of the tube body, over a tube body length from 5 mm to 500 mm, especially 10 mm to 150 mm, preferably 20 mm to 90 mm.

As a rule the flock material penetrates partially, especially only partially, into the adhesive layer.

Preferably, when an adhesive layer is present that covers the surface of the tube body at least partially, the flock material does not penetrate into the tube body or its wall.

The flock material can penetrate into the adhesive layer over a length of 0.5% to 95%, especially 3% to 50%, preferably 5% to 20%, relative to its total length.

The adhesive layer preferably is not a textile layer or coating. For example, the adhesive layer can be in the form of a film, a foil, a gel, especially hydrogel, or a paste.

In an alternative example, the adhesive layer is of a textile configuration. It is conceivable for example for the adhesive layer to be in the form of a fiber or a filament or else in the form of a large number of fibers or filaments (multifilament). In particular, the adhesive layer can be a tubular textile fabric, especially plaited tube or knitted tube, which envelops or covers the tube body. The tubular fabric can be flocked before or after fitting on the tube body (in the latter case together with the tube body). The examples described in this paragraph have the particular advantage that the tube body can be removed again without the tubular fabric from a patient's body, especially in the case when the tubular fabric is formed from an absorbable material.

In another example, the adhesive layer has a proportion of 0.1 wt % to 90 wt %, especially 0.5 wt % to 50 wt %, preferably 1 wt % to 25 wt %, relative to the total weight of the tube.

The adhesive layer can moreover have a thickness between 1 μm and 500 μm, especially 3 μm and 300 μm, preferably 5 μm and 100 μm.

Basically the adhesive layer can be configured to be absorbable, partially absorbable or non-absorbable. In other words the adhesive layer can have an absorbable, partially absorbable or non-absorbable material, preferably polymer, especially copolymer, or can be formed from such a material, preferably polymer, especially copolymer.

The term “copolymer” means, in the sense of this disclosure, a polymer that is composed of at least two different monomer units. Therefore the term “copolymer” can cover not only copolymers in the narrower sense, i.e. so-called bipolymers (polymers that are composed of two different monomer units), but in addition terpolymers, tetrapolymers etc. The copolymer can moreover be selected from the group consisting of random copolymer, alternating copolymer, block copolymer or segmented copolymer, graft copolymer and mixtures thereof.

All materials that are suitable for carrying out flocking, i.e. for joining the flock material to the adhesive layer and thus to the surface of the tube body, may basically be considered for the adhesive layer.

Thus, the adhesive layer can basically have a synthetic or industrial polymer and/or biopolymer, i.e. naturally occurring polymer, or can be formed from such a polymer and/or biopolymer.

For example, the adhesive layer can have a non-absorbable material, especially non-absorbable polymer, or can be formed from such a material, especially polymer, which is preferably selected from the group comprising hot-melt adhesives, polyurethanes, especially thermoplastic polyurethanes, polyvinyl alcohol, polyvinyl acetate, polyvinyl chloride, polyvinyl propionates, polyacrylates, cyanoacrylates, poly-α-olefins, polyurethanes, thermoplastic polyurethanes, thermoplastic polyurethane elastomers, vinyl acetate copolymers, especially modified vinyl acetate copolymers, polyesters, vinylpyrrolidone/vinyl acetate copolymer, polycarbonates, rubber, polyoxymethylene, acrylonitrile-butadiene-styrene copolymer, polyethylene glycol-based hydrogels, resins such as for example synthetic resins, waxes such as for example synthetic waxes and/or beeswaxes and mixtures thereof.

Suitable thermoplastic polyurethane elastomers are for example commercially available under the designations TEXIN® or DESMOPAN®.

Preferably the adhesive layer has a hot-melt adhesive or is formed from such an adhesive. Suitable hot-melt adhesives can be selected from the group comprising hot-melt adhesives based on polyamides, based on polyester elastomers, based on polyolefins, especially polyethylene, based on amorphous poly-α-olefins, based on polybutene-1, based on ethylene-vinyl acetate copolymer, based on polyurethane elastomers, based on copolyamide elastomers, based on vinylpyrrolidone-vinyl acetate copolymers and mixtures thereof.

Suitable resins that may be mentioned are for example resins that are selected from the group comprising urethane resins, epoxy resins, polyurethane resins, melanin resins, phenol resins, polyester resins, polyamide resins, vinyl ester resins and mixtures thereof.

Alternatively to or in combination with the non-absorbable materials, especially polymers, described in the preceding examples, the adhesive layer can have an absorbable material, especially polymer, or can be formed from such a material, especially polymer, which is preferably selected from the group comprising polyhydroxyalkanoates, polyglycolide or polyglycolic acid, polylactide or polylactic acid, polydioxanone, poly-3-hydroxybutyrate or poly-3-hydroxybutyric acid, poly-4-hydroxybutyrate or poly-4-hydroxybutyric acid, technical polysaccharides, oxidized or non-oxidized polysaccharides, polysaccharides bearing amino groups, polysaccharides bearing aldehyde groups, dextran aldehyde, cellulose, cellulose derivatives, for example alkyl celluloses, methylcellulose, hydroxyalkyl celluloses, hydroxymethylcellulose, hydroxyethylcellulose, hydroxypropylcellulose, carboxyalkyl celluloses, carboxymethylcellulose, copolymers thereof, salts thereof, stereoisomers thereof and mixtures thereof.

Alternatively to or in combination with the materials, especially polymers, stated in the preceding examples for the adhesive layer, in a further example this can have a biopolymer or can be formed from a biopolymer, which is preferably selected from the group comprising polysaccharides, mucopolysaccharides, starch, amylose, amylopectin, dextran, dextrin, cellulose, chitin, chitosan, hyaluronic acid, dextran sulphate, heparin, heparan sulphate, chondroitin sulphate, dermatan sulphate, structural proteins, extracellular proteins, fibrous proteins, globular proteins, collagen, gelatin, elastin, reticulin, fibronectin, laminin, fibrin, albumin, copolymers thereof, salts thereof, stereoisomers thereof and mixtures thereof.

Of course, the adhesive layer can also have a mixture of the materials described in the preceding examples or can consist of such a mixture.

In another example the adhesive layer can be formed from a material, especially a polymer such as for example a thermoplastic polymer, which has a lower melting point than a material, especially polymer such as thermoplastic polymer for example, from which the tube body is formed. As a result, especially advantageously a specific thermal activation of the adhesive layer can be achieved, without the integrity of the tube body structure being impaired. In particular, the melting point of the material used for making the adhesive layer can be lower than the melting point of the flock material. Suitable materials or polymers for the examples described in this paragraph are, in addition to polyethylene and/or polypropylene, basically all industrial melt adhesives or mixtures of melt adhesives. To that extent, therefore, reference is also made expressly to the preceding description.

In another example the flock material is joined to the tube body or its surface directly or without using an adhesive (or without an adhesive layer at least partially covering the surface of the tube body).

In particular, in this case the flock material penetrates directly into the wall of the tube body.

For example, the flock material can penetrate directly into the wall of the tube body over a length of 0.5% to 95%, especially 3% to 50%, preferably 5% to 20%, relative to its total length.

The tube body, especially one or optionally several surface layers thereof, preferably has a material, especially polymer, or is formed from a material, especially polymer, which can by activation be transformed into an adhesive or sticky state. Suitable activation techniques can be selected from the group comprising melting, liquefying, moistening, impregnating, dipping, heating, irradiating, production of ultrasonic waves, the use of chemicals and combinations thereof.

The flock material can basically be in the form of a dust, powder or granules.

Preferably the flock material is in the form of fibers, especially cut fibers and/or ground fibers.

In other words it is preferable if the flock material is flock fibers, especially cut flock fibers and/or ground flock fibers.

In particular, the flock material can be textured and/or non-textured flock fibers.

In another example the flock material is branched flock fibers. The use of branched flock fibers has the advantage that the tube body basically has to be flocked with fewer flock fibers in order to produce a relatively large volume, in particular that fills body lumina or cavities.

The flock material, especially in the form of fibers, can have a length of 1 mm to 35 mm, especially 2 mm to 20 mm, preferably 3 mm to 10 mm.

Furthermore, the flock material, especially in the form of fibers, can have a diameter from 5 μm to 800 μm, especially 7 μm to 300 μm, preferably 10 μm to 150 μm.

Moreover, the flock material, especially in the form of fibers, can have a titre (weight per unit length) from 0.1 dtex to 350 dtex, especially 0.5 dtex to 100 dtex, preferably 1 dtex to 80 dtex. The dimension “dtex” (decitex) means a weight per unit length of 1 g per 10000 m length of the flock material.

In another example, the flock material, especially in the form of fibers, can project over a length from 0.01 mm to 14 mm, especially 0.1 mm to 9.0 mm, preferably 0.2 mm to 4.8 mm, from the surface of the tube body, especially from the adhesive layer at least partially covering the surface of the tube body.

Furthermore, the flock material, especially in the form of fibers, can protrude perpendicularly or essentially perpendicularly from the surface of the tube body, especially the adhesive layer at least partially covering the surface of the tube body. The expression “essentially perpendicularly” can comprise deviations from right angles by up to 5 degrees.

In an alternative example the flock material, especially in the form of fibers, protrudes at an oblique angle from the surface of the tube body, especially the adhesive layer at least partially covering the surface of the tube body, which is greater than 0 degree, but less than 85 degrees.

In another example, the flock material, especially in the form of flock fibers, is disposed at a density from 1% to 30%, especially 5% to 25%, preferably 10% to 20%, on the surface of the tube body, especially the adhesive layer at least partially covering the surface of the tube body, relative to the theoretical flock material density (coverage density). The term “theoretical flock material density (coverage density)” means, in the sense of this disclosure, a value that corresponds to the square of flock materials arranged seamlessly next to one another, especially in the form of flock fibers, on one millimetre divided by the fiber diameter.

In another example a flock material in the form of fibers is disposed at a density from 1 to 2000 fibers/mm², especially 5 to 1000 fibers/mm², preferably 10 to 500 fibers/mm², on the surface of the tube body, especially the adhesive layer at least partially covering the surface of the tube body.

The flock material can basically, apart from any openings in the wall of the tube body, be disposed on part of the area or on the whole area of the surface of the tube body, especially the surface of the adhesive layer covering the tube body at least partially.

In desirable examples, the flock material is disposed at least on one of the ends of the tube body, especially on only one of the ends of the tube body, on the surface of the tube body, especially on the adhesive layer at least partially covering the surface of the tube body.

In a further example, the flock material, preferably starting from at least one of the ends of the tube body, is disposed over a tube body length from 5 mm to 1000 mm, especially 5 mm to 500 mm, preferably 10 mm to 150 mm, especially preferably 20 mm to 90 mm, on the surface of the tube body, especially on the adhesive layer at least partially covering the surface of the tube body.

The flock material can in principle be arranged in the form of a pattern or irregularly or randomized on the tube body, especially on the adhesive layer at least partially covering the surface of the tube body.

Suitable arrangements of flock material (and/or arrangements of adhesive layer) on the tube body, especially the adhesive layer at least partially covering the surface of the tube body, can be selected from the group comprising linear arrangement, staggered arrangement, helical or spiral arrangement, meander-like arrangement, serpentine arrangement, sinusoidal arrangement, arrangement as circular rings and combinations thereof.

It is preferable if the flock material is arranged in spirals or circular rings on the surface of the tube body, especially the adhesive layer at least partially covering the surface of the tube body.

A helical or spiral arrangement of the flock material can be achieved for example by covering the surface of the tube body with a helically or spirally wound filament, serving as adhesive. Alternatively, a helical or spiral arrangement of the flock material can be achieved by means of plaiting acting as adhesive, the filaments of which are partially formed from a thermoplastic polymer with a lower melting point than other filaments of the plaiting.

The possible arrangements of the flock material described in the preceding examples can especially advantageously serve as fixing, monitoring, marking, reference or stop or holding means.

The flock material itself can basically be absorbable, partially absorbable or non-absorbable. In other words the flock material can have an absorbable, partially absorbable or non-absorbable material, preferably polymer, especially copolymer, or can be formed from such a material, preferably polymer, especially copolymer.

Thus, flock material can basically have a synthetic or industrial polymer and/or biopolymer, i.e. naturally occurring polymer, or can be formed from such a polymer and/or biopolymer.

Preferably the flock material has a polymer or is formed from a polymer that is selected from the group comprising polyolefins, polyamides, polyesters, polyurethanes, especially thermoplastic polyurethanes, polyhydroxyalkanoates, polysaccharides, technical polysaccharides, oxidized and non-oxidized polysaccharides, polysaccharides bearing amino groups, polysaccharides bearing aldehyde groups, mucopolysaccharides, proteins, structural proteins, extracellular proteins, fiber proteins, globular proteins, enzymes, antibodies, blood clotting factors, copolymers thereof, salts thereof, stereoisomers thereof and mixtures thereof.

For example the flock material can be selected from the group comprising polyethylene, low-density polyethylene, high-density polyethylene, high-molecular polyethylene, ultra-high-molecular polyethylene, polypropylene, polyethylene terephthalate, polypropylene terephthalate, polybutylene terephthalate, polyacrylonitrile, polyamide 6, polyamide 6-6, polyamide 6-12, polyamide 12, silk, especially rayon or spider silk, polytetrafluorethylene, polyvinylidene difluoride, polytetrafluoropropylene, polyhexafluoropropylene, polyvinyl alcohol, polyglycolide or polyglycolic acid, polylactide or polylactic acid, polydioxanone, poly-3-hydroxybutyrate or poly-3-hydroxybutyric acid, poly-4-hydroxybutyrate or poly-4-hydroxybutyric acid, polytrimethylene carbonate, poly-ε-caprolactone, cotton, cellulose, cellulose derivatives such as for example alkyl celluloses, methylcellulose, hydroxyalkyl celluloses, hydroxymethylcellulose, hydroxyethylcellulose, hydroxypropylcellulose, carboxyalkyl celluloses, carboxymethylcellulose, starch, amylose, amylopectin, dextran, dextrin, chitin, chitosan, hyaluronic acid, dextran sulphate, heparin, heparan sulphate, chondroitin sulphate, dermatan sulphate, collagen, gelatin, elastin, reticulin, fibronectin, laminin, fibrin, fibrinogen, albumin, copolymers thereof, salts thereof, stereoisomers thereof and mixtures thereof.

A possible copolymer for the flock material is for example a copolymer of glycolide and lactide, especially in a weight ratio from 9:1 to 1:9, especially 7:3 to 3:7.

A possible terpolymer, especially triblock terpolymer, for the flock material is for example a terpolymer, especially triblock terpolymer, of glycolide, trimethylene carbonate and ε-caprolactone. A terpolymer of this kind is commercially available for example under the designation MONOSYN®.

In another example, the flock material has a material that is swellable or soluble in aqueous liquids, especially aqueous solutions, aqueous suspensions, aqueous buffer solutions, aqueous electrolyte solutions and/or body fluids, or in another example is formed from such a material. A suitable material is preferably selected from the group comprising polyvinyl pyrrolidone, polyvinyl alcohol, polyethylene glycol or polyethylene glycol-based copolymers, viscose, cotton, cellulose, cellulose derivatives such as for example alkyl celluloses, methylcellulose, hydroxyalkyl celluloses, hydroxymethylcellulose, hydroxyethylcellulose, hydroxypropylcellulose, carboxyalkyl celluloses, carboxymethylcellulose, starch, amylose, amylopectin, dextran, dextrin, chitin, chitosan, hyaluronic acid, dextran sulphate, heparin, heparan sulphate, chondroitin sulphate, dermatan sulphate, collagen, gelatin, elastin, reticulin, fibronectin, laminin, fibrin, fibrinogen, albumin, copolymers thereof, salts thereof, stereoisomers thereof and mixtures thereof.

The flock material, in particular a water-soluble flock material, especially as described in the preceding paragraph, is suitable in particular for having pharmaceutical or medical active substances added, which will be discussed in more detail below. These active substances can then especially advantageously be released during therapy. In this way the tube can be used as a drug delivery device.

In another example the flock material has a physiologically or biologically active material or is formed from such a material. The expression “physiologically active material” or “biologically active material” means a material which, from the cosmetic and/or medical standpoint, can produce advantageous effects in a patient's body.

In particular, the flock material can have a cosmetic active substance, biological active substance, pharmaceutical or medical active substance or mixtures thereof or can be formed from said active substance or a mixture of said active substances.

For example the flock material can have an active substance or can be formed from an active substance that is selected from the group comprising antimicrobial, especially antibiotic, active substances, active substances promoting wound healing, disinfecting active substances, skin-care active substances, oncologic active substances, scar-treating or scar-preventing active substances, anti-inflammatories, pain-relieving active substances, active substances promoting blood coagulation, growth factors, cell differentiation factors, cell-adhesion factors, cell-recruiting factors, cell receptors, cell binding factors, cytokines, peptides, structural proteins, extracellular proteins such as for example collagen, polysaccharides such as for example hyaluronic acid, oligonucleotides, polynucleotides, DNA, RNA, salts thereof, stereoisomers thereof and mixtures thereof.

In particular the flock material can have an active substance or can be formed from an active substance that is selected from the group comprising FGF (fibroblast growth factor), TGF (transforming growth factor), PDGF (platelet-derived growth factor), EGF (epidermal growth factor), GMCSF (granulocyte-macrophage colony stimulation factor), VEGF (vascular endothelial growth factor), IGF (insulin-like growth factor), HGF (hepatocyte growth factor), IL-1B (interleukin-1-B), IL-8 (interleukin-8), NGF (nerve growth factor), BMP or bone morphogenetic proteins such as for example BMP-1 (bone morphogenetic protein 1) and/or BMP-2 (bone morphogenetic protein 2), salts thereof, stereoisomers thereof and mixtures thereof.

Suitable antimicrobial active substances that may be mentioned are for example active substances that are selected from the group comprising biguanides, polyhexamethylene biguanide (PHMB), triclosan, chlorhexidine, gentamicin, copper, zinc, silver, gold, salts thereof, stereoisomers thereof and mixtures thereof.

A preferred active substance promoting wound healing is for example zinc, especially a zinc pigment. Zinc-containing, especially zinc pigment containing, cellulose fibers are especially preferred.

A preferred scar-treating or scar-preventing active substance is captopril.

A suitable anti-inflammatory is for example the pharmaceutical called ibuprofen.

A suitable pain-relieving active substance that may be mentioned is for example the pharmaceutical called paracetamol.

A suitable active substance promoting blood coagulation is for example para-aminomethylbenzoic acid, available commercially under the designation PAMBAL®.

Of course, the flock material can also have a mixture of the materials described in the preceding examples or can consist of such a mixture.

In another example, the flock material has a proportion from 0.01 wt % to 50 wt %, especially 0.5 wt % to 20 wt %, preferably 1.0 wt % to 10 wt %, relative to the total weight of the tube.

In another example the flock material is a mixture of at least two different flock fibers. The flock fibers are preferably varied with respect to their flock fiber length, their flock fiber diameter, their flexibility or stiffness, their material, their absorbability or non-absorbability, their solubility, their structure and/or functionalization, especially with additives, for example with active substances. Regarding the properties of the flock fibers listed above as examples, reference is made entirely to the description up to now.

It can, for example, be envisaged that the flock material is in the form of a mixture of flock fibers with different stiffness or different flexibility. For example the flock material can be a mixture of flock fibers made from polyamide 6 and flock fibers made from polyvinyl alcohol. Whereas the flock fibers of polyamide 6, which is practically unswellable in water, preferably serve as spacers (between tissue and tube body) during drainage, the flock fibers of polyvinyl alcohol preferably act as a sliding agent.

Furthermore, the flock material can be a mixture of textured and non-textured flock fibers.

Furthermore, it can be envisaged that the flock material comprises water-soluble or water-swellable flock fibers in addition to water-insoluble flock fibers, wherein the water-soluble or water-swellable flock fibers preferably are longer than the water-insoluble flock fibers. The water-soluble or water-swellable flock fibers act especially advantageously as a sliding agent, making the additional use of a lubricant unnecessary. Moreover, through the presence of water-soluble or water-swellable flock fibers, the imperviousness of the flock material can be modulated. Cotton and/or viscose fibers are especially preferred. Moreover, regarding suitable materials for the flock fibers described in this paragraph, especially for the water-soluble or water-swellable flock fibers, reference is made entirely to the description so far.

Furthermore, it can be envisaged that the flock material comprises predetermined breaking points. The predetermined breaking points can be selected from the group comprising mechanical predetermined breaking points, chemical predetermined breaking points and combinations thereof. For example, mechanical predetermined breaking points can be accomplished by thinning out the flock material, in particular if the flock material is present in form of fibers. Chemical predetermined breaking points can be, for example, accomplished by structural changes from crystalline to amorphous structure and vice versa. In general, the presence of predetermined breaking points can facilitate removal of the medical tube from a patient's body and can additionally accelerate resorption of the flock material.

In advantageous examples the tube body is formed from a material that is at least partially swellable or partially soluble in a solvent, preferably organic solvent such as for example ethyl acetate, tetrahydrofuran (THF), hexane, acetone, aryl compounds, especially toluene and/or xylene, dichloromethane, chloroform or mixtures thereof. As a result, the tube body, especially at least one or more surface layers thereof, can act as adhesive for the flock material. The presence of a separately formed adhesive layer at least partially covering the surface of the tube body is therefore unnecessary.

In another advantageous example the tube body is formed from a flexible material, for example silicone. As a result, in particular flexible adjustment of the cross-section of the tube body (and thus of the tube) to its surroundings is possible. Moreover, this facilitates removal of the tube from a patient's body.

Preferably the tube body has a material or is formed from a material that is selected from the group comprising polyvinyl chloride, polyurethane, polyethylene, polypropylene, silicone and mixtures thereof. The use of polyvinyl chloride as material for the tube body has the advantage that a correspondingly produced tube body can be solubilized with THF and in this way can be made adhesive or sticky (for flocking). A corresponding effect can be achieved with a tube body made of polyurethane, by solubilizing with dichloromethane and/or chloroform. A tube body made of polyethylene can for example be transformed into an adhesive or sticky state suitable for flocking by solubilizing with acetone. A tube body made of polypropylene can be made adhesive or sticky for flocking for example by solubilizing with xylene.

The tube body can have an outside diameter from 1 mm to 15 mm, preferably 2 mm to 10 mm, more preferably 3 mm to 8 mm, especially preferably 3 mm to 7 mm, most preferably 4 mm to 6 mm.

Furthermore, the tube body can be of multilumen configuration, i.e. can possess two, three or more lumina (cavities). The lumina can in particular have different cross-sections.

For example, the tube body can be of three-lumen configuration, wherein one lumen is provided for applying a negative pressure or vacuum, one lumen for rinsing and one lumen for monitoring or control of a negative pressure or vacuum and/or a temperature. Temperature monitoring can provide measurement or monitoring of the inflammatory state and therefore the progress of healing.

In particular, the tube can comprise several tube bodies that are joined together, for example glued together, and all tube bodies or just some of the tube bodies can be flocked with the flock material.

In an alternative example, the tube can comprise several single, i.e. separately configured, tube bodies. All tube bodies or just some of the tube bodies can be flocked with the flock material.

In a further example, the tube body can be configured as a branched tube body. For example, the tube body can comprise two, three or more branches, with all branches or just some of the branches being flocked with the flock material. Thus, blockage of one branch can be advantageously compensated by the remaining branches without need of interrupting therapy of a patient.

For leading away body fluids or for fluid communication between the surroundings of the tube and a lumen of the tube body it is envisaged in preferred examples that the tube body has openings in its wall, especially in the form of holes, perforations, pores, cut-outs or the like. The openings are as a rule configured in the form of through-holes, i.e. as a rule they breach the wall of the tube body. Preferably the tube body has corresponding openings in the region of at least one of its ends, especially in the region of only one of its ends.

Furthermore, the tube body can have protuberances, especially for increasing its surface area.

In another example, the tube body has a circular cross-section or non-circular cross-section, especially an oval, ellipsoidal, crescent-shaped, polygonal for example triangular, rectangular, square, rhombus-shaped, pentagonal, hexagonal or star-shaped cross-section.

In particular, the cross-section of the tube body can be variable over its length.

For example, the tube body can be configured as conical or as a tapered section.

In further examples, the tube, especially the tube body and/or the flock material, has additives such as in particular physiologically compatible materials, preferably cosmetic, biological and/or medical or pharmaceutical active ingredients, X-ray contrast media, radio-opaque substances, textile auxiliaries such as for example guide aids or spiral mandrin or the like. Regarding suitable physiologically compatible materials or active substances, reference is made entirely to the description so far.

As a supplement or alternative, the tube, especially the tube body and/or the flock material, can have cells. Suitable cells can be selected from the group comprising fibroblasts, chondrocytes, osteocytes, osteoblasts, adipocytes, myocytes, neurons, astrocytes, oligodendrocytes, hepatocytes, pancreatic cells, precursor cells thereof, stem cells, especially mesenchymal stem cells, adult stem cells, umbilical cord stem cells and/or fetal stem cells, and mixtures thereof. By using cells or enriched cells, for example the progress of wound healing can be accelerated.

Furthermore, the cells can be autologous, allogenous and/or xenogenous cells. To avoid immune reactions, cells of autologous origin are basically preferred.

To supplement or as an alternative to the example described in the three preceding paragraphs, the tube according to this disclosure, especially the tube body and/or the flock material, can have platelet-rich plasma (PRP).

In a further example, the tube can be present in a compressed state due to a preferably dissolvable wrapping or impregnation. The wrapping and impregnation, respectively can be formed of a protein such as for example collagen and/or gelatine and/or a polysaccharide such as for example starch and/or hyaluronic acid.

Though sliding properties of the tube can be targeted by choice of the flock material, it can be further envisaged that the tube comprises a slide coating. Thus, the sliding properties of the tube can be additionally improved. The slide coating can be formed of a protein and/or polysaccharide, in particular selected from the group comprising collagen, gelatine, starch, hyaluronic acid and combinations thereof.

Regarding further features and advantages of the tube, especially of tube body and/or of flock material, reference is made entirely to the description given hereunder.

In a second aspect, this disclosure relates to a process for producing a medical tube, preferably a tube such as has been described in the preceding examples.

The process comprises the steps:
a) at least partial, especially partial or complete, coating of the surface of at least one tube body with an adhesive and
b) flocking the adhesive-coated tube body with a flock material, while the adhesive is moist, adhesive or sticky, or after activating the adhesive.

The term “flocking” means, in the sense of this disclosure, a technique for applying a flock material, especially in the form of fibers, on the tube body (as substrate) or the adhesive layer at least partially covering the surface of the tube body.

The term “activating” or “activate” should comprise, in the sense of this disclosure, a technique by means of which the adhesive can be made moist, adhesive or sticky. Corresponding techniques can be selected from the group comprising melting, liquefying, moistening, impregnating, dipping, heating, irradiating, production of ultrasonic waves, the use of chemicals and combinations thereof.

In order to be able to flock the tube body with the maximum possible amount of flock material or to produce a high density of flock material on the tube body, in desirable examples a larger tube body is used for producing the tube than the tube bodies described hitherto in the prior art in connection with vacuum therapy. Regarding suitable outside diameters for a tube body for producing the tube of this disclosure, reference is made to the outside diameters disclosed in the context of the first aspect of this disclosure.

In a preferred example, the tube body is provided, before or after flocking, preferably before flocking, with openings, especially in the form of holes, pores, cut-outs, perforations or the like, as a rule with penetration through the tube body wall.

The tube body can be coated with the adhesive for example by extrusion, injection, moulding, screen-printing, dipping, impregnating, spraying, spreading, knife-coating and/or rolling techniques.

The coating techniques described above depend in particular on the adhesive concretely used.

If for example the adhesive is formed from a fusible material, the adhesive can be applied on the tube body by extrusion techniques. If, however, the adhesive is in the form of an aqueous liquid, especially aqueous solution, it may be preferable to apply the adhesive on the tube body by means of a dipping, spraying or spreading technique.

If the adhesive is in the form of paste or gel, especially hydrogel, it may be advantageous to apply the adhesive on the tube body by brushing, spreading, knife-coating, rolling or the like.

The flocking of the tube body can basically be carried out by means of scattering techniques, spraying techniques, blowing techniques, vibration techniques and/or electrostatic techniques.

Preferably the flocking of the tube body is carried out by means of electrostatic flocking.

In electrostatic flocking, the flock material is applied in an electric field on the tube body or the adhesive layer at least partially covering the surface of the tube body.

In desirable examples, electrostatic flocking is carried out in a flocking machine. As a rule the flocking machine receives a negative charge, while as a rule the tube body is grounded. As a result the flock material is usually “shot” perpendicularly or essentially perpendicularly onto the tube body or the adhesive layer at least partially covering the surface of the tube body.

Owing to the high acceleration that the flock material experiences in the electrostatic field, as a rule good penetration of the flock material into the tube body or the adhesive layer at least partially covering the surface of the tube body is achieved.

In preferred examples the tube is used as electrode, especially grounded electrode, together with a counter-electrode, especially a high-voltage electrode. Especially preferably, the tube body is used as cathode and the counter-electrode, preferably a high-voltage electrode, is used as anode.

The electrodes (tube body and counter-electrode) can be spaced from 2 cm to 200 cm apart, especially 5 cm to 100 cm, preferably 7 cm to 50 cm.

Furthermore, for carrying out electrostatic flocking, a voltage from 5 kV (kilovolt) to 120 kV (kilovolt), especially 10 kV (kilovolt) to 100 kV (kilovolt), preferably 20 kV (kilovolt) to 80 kV (kilovolt), can be applied between the electrodes.

After flocking and especially before a drying step, it can be envisaged according to this disclosure to modify the flocked tube body, especially the flock material, in order to achieve particular effects. For example, the flocked tube body, especially the flock material, can be modified with respect to orientation, stiffness, shape, cross-section, possible crimping and/or length. This can provide indication-specific and/or patient-specific adaptation of the flock material (and thus of the tube). Corresponding modifications can be achieved for example by means of heat, welding, especially thermal welding, chemicals or the like.

To improve its sliding properties, hydrophilization of the tube, especially of the tube body and/or the flock material, can further be envisaged. Alternatively, to improve its sliding properties the tube can also be lipophilized, which can be achieved for example through an appropriate choice of the flock material or (subsequent) modification of the flock material.

Regarding further features and advantages of the process described in the preceding examples, especially of the tube, tube body and/or of the flock material, reference is made entirely to the examples made in the context of the first aspect of this disclosure.

In a third aspect, this disclosure provides an alternative process for producing a medical tube, preferably as described in the context of the first aspect of this disclosure. This process comprises the steps:
a) at least partial transformation of at least one tube body into an adhesive or sticky state and
b) flocking of the at least partially adhesive or sticky tube body with a flock material.

The tube body can be transformed into a partially adhesive or sticky state for example by means of melting, liquefying, moistening, impregnating, dipping, heating, irradiating, production of ultrasonic waves, the use of chemicals or combinations thereof.

In desirable examples the tube body is only transformed partially into an adhesive or sticky state.

Preferably one or more surface layers of the tube body are transformed into an adhesive or sticky state.

Especially preferably the tube body, especially one or more surface layers thereof, is transformed into an adhesive or sticky state by solubilizing with a solvent, preferably an organic solvent. Suitable solvents can be selected from the group comprising ethyl acetate, tetrahydrofuran (THF), hexane, acetone, aryl compounds, especially toluene, xylene and mixtures thereof, dichloromethane, chloroform and mixtures thereof.

Regarding further features and advantages of the process, reference is made entirely to the description so far, i.e. to the account provided in the context of the first and second aspect of this disclosure.

The processes described in the context of the second and third aspect of this disclosure can basically be intended for the production of a medical tube that is selected from the group comprising drainage tube, rinsing tube, tube for monitoring or control of negative pressure or vacuum, for monitoring a temperature and/or for administering therapeutic agents and/or disinfectants or the like.

In especially preferred examples, the processes described in the context of the second and third aspect of this disclosure are intended for production of a medical drainage tube. Correspondingly, the at least one tube body is preferably at least one drainage tube body.

In a fourth and last aspect, this disclosure relates to a drainage system, especially in the form of a kit. The drainage system is intended in particular for use in vacuum therapy, preferably endoluminal vacuum therapy.

The drainage system is characterized in particular in that it has at least one medical tube, preferably as disclosed in the description so far, and additionally at least one further component, wherein the further component is selected from the group comprising sponge, inserting instrument, for example overtube or trocar, source of vacuum or negative pressure, redrop bottle, guide wire, spiral mandrin and combinations thereof.

The medical tube can basically be selected from the group comprising drainage tube, rinsing tube, tube for monitoring or control of negative pressure or vacuum, monitoring a temperature and/or for administering therapeutic agents, disinfectants or the like.

In especially preferred examples, the medical tube of this disclosure is a medical drainage tube.

The use of a sponge may be advantageous for sealing or closing off a body orifice during a drainage operation. Moreover, the sponge can also be flocked, i.e. comprise a flock material, especially a flock material with antibacterial additive. Regarding further features and advantages of the flock material and a possible adhesive layer for joining the flock material to the sponge, reference is made entirely to the description so far.

Regarding further features and advantages of the drainage system, especially of the medical tube, reference is made entirely to the description so far.

The objects discussed in the preceding aspects of this disclosure (tube, production processes and drainage system) can also each be used for topological applications, especially for topological vacuum therapy.

Further features and advantages can be seen from the following description of preferred examples in the form of drawings, descriptions of drawings and practical examples. Moreover, individual features can in each case be realized individually or in combination with one another. The preferred examples only serve for further explanation and better understanding of this disclosure, without limiting it to these.

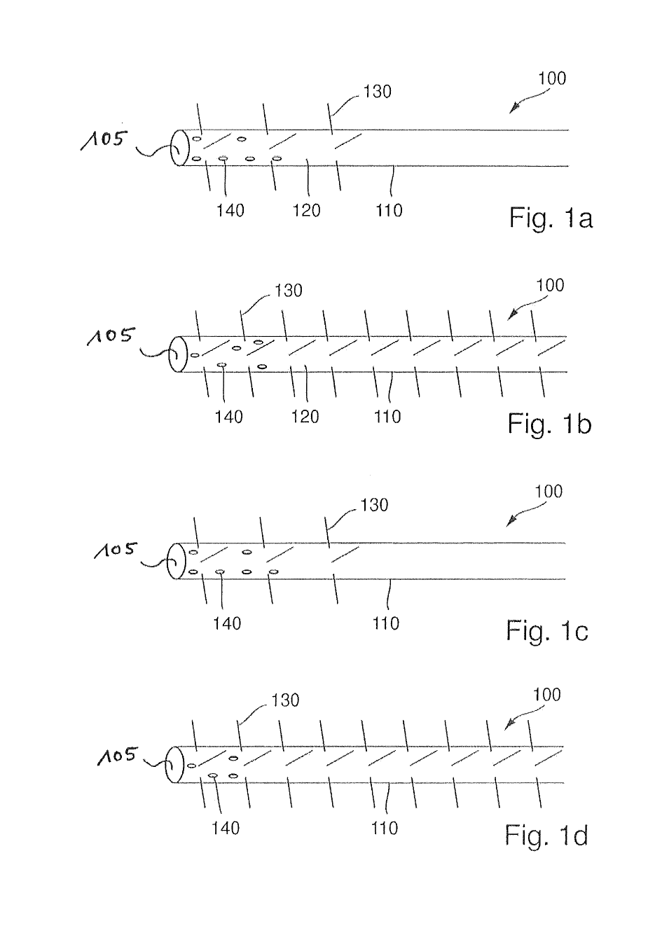

FIG. 1a shows schematically a medical tube 100. This comprises a tube body 110, an adhesive layer 120 and a flock material 130 in the form of fibers (flock fibers). The tube body 110 has a lumen (cavity) 105. The flock fibers can for example be formed from a polyamide (nylon flock fibers). Moreover, the flock fibers can for example contain silver as additive for achieving antimicrobial functionality.

The adhesive layer 120 covers the outside surface of the tube body 110 only on one of its two ends. The flock fibers 130 are connected via the adhesive layer 120 to the tube body 110 or its outside surface and protrude from the adhesive layer 120.

For leading away body fluids, especially wound fluids, blood, lymph or the like, the tube body 110 can moreover have openings 140 in the region of its flocked end.

FIG. 1b shows schematically another example of a tube 100. The tube 100 has a tube body 110 with a lumen (cavity) 105, an adhesive layer 120 and a flock material 130 in the form of fibers (flock fibers) and is flocked in its entirety or on the whole area with the flock fibers 130.

In contrast to the tube shown in FIG. 1a, the adhesive layer 120 covers the outside surface of the tube body 110 continuously or on the whole area (apart from optional openings 140). Correspondingly, the tube body 110 or its outside surface is flocked continuously (apart from optional openings 140) with the flock material 130.

The tube 100 shown in FIG. 1b can for example be placed in the form of a “ball” in a body cavity, so that optimum filling of the cavity with the tube 100 can be achieved.

FIG. 1c shows schematically another example of a tube 100. This has a tube body 110 with a lumen (cavity) 105 and a flock material 130 in the form of fibers (flock fibers). The fibers 130 can for example be polyurethane fibers.

The fibers 130 are only arranged, in the region of one of the two ends of the tube body 110, on the latter or its outside surface.

For leading away body fluids, especially wound fluids, blood, lymph or the like, the tube body 110 can moreover have openings 140 in the region of its flocked end.

In contrast to the tubes shown in FIGS. 1a and 1b, the flock material 130 is connected directly, i.e. without the use of an adhesive layer covering the outside surface of the tube body, to the tube body 110 or its outside surface. Rather, the flock material 130 penetrates directly into the wall of the tube body 110. A tube body suitable for this can be formed for example from polyvinyl chloride or polyurethane.

FIG. 1d shows schematically another example of a tube 100. This has a tube body 110 with a lumen (cavity) 105 and a flock material 130 in the form of fibers (flock fibers), for example in the form of viscose fibers.

For leading away body fluids, the tube body 110 can also have openings 140 in the region of its flocked end.

In contrast to the tube shown in FIG. 1c, the tube body 110 or its outside surface is flocked continuously or on the whole area (apart from possible openings 140) with the flock material 130.

The tube 100 shown in FIG. 1d can also be placed for example in the form of a “ball” in a body cavity, so that optimum filling of the cavity with the tube 100 can be achieved.

The tubes shown in FIGS. 1a-d can preferably be used as drainage tubes, especially for carrying out vacuum therapy, preferably endoluminal vacuum therapy. In this case it may furthermore be preferable if one of the tube ends, especially the flocked tube end (of the tubes shown in FIGS. 1a-d), is completely sealed, for example glued or welded. This can, especially advantageously, increase the suction power or the suction capacity of the drainage tubes.

Alternatively, the tubes shown in FIGS. 1a-d can be used for introducing a rinsing fluid, supplying therapeutic agents, monitoring or control of a negative pressure/vacuum and/or for temperature monitoring. In these cases it may be desirable if the tube body does not have any openings, especially in the form of holes, pores, cut-outs or the like.

### EXAMPLES

### Example 1

The perforated (with holes with a diameter of 1 mm) end of a polyurethane tube with an outside diameter of 5 mm and an inside diameter of 3 mm was dipped over a length of 6 cm in a THF bath for 5 seconds. Then the polyurethane tube solubilized in this way was immediately introduced into a flocking booth and was flocked by applying a high voltage of 60 kV (kilovolt) with polyurethane flock fibers with a length of 5 mm (30 dtex). The polyurethane flock fibers were then applied by means of a sieving-dosing applicator on the outside surface of the polyurethane tube, wherein the sieving-dosing applicator was arranged at a distance of 40 cm from the polyurethane tube. Then the resultant flocked polyurethane tube was dried for 6 hours at a temperature of 80° C. Polyurethane flock fibers that were not adhesively bonded were removed by means of a vacuum suction device.

### Example 2

In the context of a continuous flocking process, an endless tube made of polyurethane was fed at a speed of 1 cm/s through a flocking machine, which had a gluing zone, a sieving-dosing unit, a drying zone and a vacuum zone. In the gluing zone, the polyurethane tube was coated in its entirety at a distance of 100 cm in each case over a tube length of 10 cm by means of a paint brush with an adhesive that is commercially available under the designation Mecoflock L 856. On entering the sieving-dosing unit, the tube segments coated with the adhesive were in each case flocked at a high voltage of 60 kV (kilovolt) with polyamide flock fibers. The distance from the tube segments to be flocked to the sieving-dosing applicator was also 40 cm. The resultant flocked endless tube was dried at a temperature between 60 and 90° C. in the drying zone. In the vacuum zone that came next, flock fibers not adhesively bonded were removed by applying a negative pressure or vacuum.

### Example 3

A tube made of polyvinyl chloride (without drainage holes) was dipped over a tube length of 40 cm several times in a melt of poly-4-hydroxybutanoic acid, until the outside surface of the tube was covered continuously with a uniform layer of poly-4-hydroxybutanoic acid.

To activate the layer of poly-4-hydroxybutanoic acid, a flocking booth was heated by means of infrared lamps to a temperature above 75° C. After the layer of poly-4-hydroxybutanoic acid had been partially fused in this way, the polyvinyl chloride tube was flocked with flock fibers made of polyglycolic acid by applying a high voltage of 60 kV (kilovolt) for 3 minutes. The distance between the flock applicator and the polyvinyl chloride tube was 40 cm. After flocking, the flocking booth was ventilated with cold ambient air. Flock fibers not adhesively bonded were then removed by means of a fan (after a cooling phase of 15 minutes).

1. A medical tube comprising at least one tube body and a flock material.

2. The medical tube according to claim 1, wherein the tube is a medical drainage tube.

3. The medical tube according to claim 1, wherein the flock material is joined to the tube body or its surface via an adhesive layer covering the surface of the tube body at least partially-or only partially.

4. The medical tube according to claim 1, wherein the adhesive is selected from the group consisting of hot-melt adhesives, hot activatable adhesives, polyvinyl alcohol, polyvinyl acetate, polyvinyl chloride, polyvinyl propionate, polyacrylates, cyanoacrylates, modified vinyl acetate copolymers, polyester elastomers, polyurethane elastomers, vinylpyrrolidone/vinyl acetate copolymer, polycarbonates, rubber, acrylonitrile-butadiene-styrene copolymers, hydrogels based on polyethylene glycol, resins, waxes and mixtures thereof.

5. The medical tube according to claim 1, wherein the flock material is joined to the tube body or its surface directly or without an adhesive layer at least partially covering the surface of the tube body.

6. The medical tube according to claim 1, wherein the flock material is arranged on the surface of the tube body on at least one of the ends of the tube.

7. The medical tube according to claim 1, wherein the flock material is flock fibers.

8. The medical tube according to claim 1, wherein the flock material is a mixture of at least two different flock fibers, wherein the flock fibers vary with respect to at least one of their flock fiber length, their flock fiber diameter, their flexibility or stiffness, their material, their absorbability or non-absorbability, their solubilities, their structure, their functionalization, or addition of active substances.

9. The medical tube according to claim 1, wherein the flock material comprises water-soluble flock fibers in addition to water-insoluble flock fibers.

10. The medical tube according to claim 1, wherein the flock material is selected from the group consisting of polyolefins, polyamides, polyimides, polyesters, polyurethanes, polyhydroxyalkanoates, proteins, polysaccharides, polyethylene, low-density polyethylene, high-density polyethylene, high-molecular polyethylene, ultra-high-molecular polyethylene, polypropylene, polyethylene terephthalate, polypropylene terephthalate, polybutylene terephthalate, polyamide 6, polyamide 6-6, polyamide 6-12, polyamide 12, silk, polytetrafluorethylene, polyvinylidene difluoride, polytetrafluoropropylene, polyhexafluoropropylene, polyvinyl alcohol, polyglycolide, polylactide, polydioxanone, poly-3-hydroxybutyrate, poly-4-hydroxybutyrate, polytrimethylene carbonate, poly-ε-caprolactone, collagen, gelatin, elastin, reticulin, fibronectin, laminin, fibrin, fibrinogen, albumin, starch, amylose, amylopectin, dextran, viscose, cotton, cellulose, methylcellulose, carboxymethylcellulose, chitosan, hyaluronic acid, dextran sulphate, heparin, heparan sulphate, chondroitin sulphate, dermatan sulphate, copolymers thereof, salts thereof, stereoisomers thereof and mixtures thereof.

11. The medical tube according to claim 1, wherein the flock material is provided with an additive selected from the group consisting of antimicrobial, active substances, disinfecting active substances, active substances promoting wound healing, anti-inflammatories, pain-relieving active substances, oncologic active substances, growth factors, cell differentiation factors, cell-adhesion factors, cell-recruiting factors, salts thereof, stereoisomers thereof and mixtures thereof.

12. The medical tube according to claim 1, wherein the tube body has an outside diameter from 1 mm to 15 mm.

13. The medical tube according to claim 1, wherein the tube body is formed from a material that is at least partially swellable or partially soluble in a solvent.

14. The medical tube according to claim 1, wherein the tube body is formed from a material that is selected from the group comprising polyvinyl chloride, polyurethane, polyethylene, polypropylene, silicone and mixtures thereof.

15. The medical tube according to claim 1, wherein the tube body is of multilumen configuration.

16. The medical tube according to claim 1, wherein the tube body has openings, especially in the form of holes, pores, perforations, cut-outs or the like.

17. A process for producing a medical tube according to claim 1, comprising the steps:
a) coating the surface of at least one tube body with an adhesive at least partially, especially partially or completely, and
b) flocking the adhesive-coated tube body with a flock material, while the adhesive is moist, adhesive or sticky, or after activating the adhesive.

18. A process for producing a medical tube according to claim 1, comprising the steps:
a) at least partial transformation of at least one tube body into an adhesive or sticky state and
b) flocking of the at least partially adhesive or sticky tube body with a flock material.

19. The process according to claim 17, wherein the tube body is provided with openings, especially in the form of holes, pores, perforations, cut-outs or the like.

20. A drainage system comprising at least one medical tube according to claim 1 and at least one further component that is selected from the group consisting of sponge, inserting instrument, source of vacuum or negative pressure, redrop bottle, guide wire, spiral mandrin and combinations thereof.
